(19) United States
(12) Patent Application Publication
(10) Pub. No.: US 2006/0263414 A1
(43) Pub. Date: Nov. 23, 2006

(54) Confectionery products for the treatment of dry mouth

(76) Inventors: Pauline Pan, Denville, NJ (US); Magdy Abdel-Malik, Chester, NJ (US)

Correspondence Address: Darryl C. Little;Warner Lambert Company LLC, 201 Tabor Road, Morris Plains, NJ, 07950, US

(21) Appl. No.: 11/418,735

(22) Filed: May 5, 2006

Related U.S. Application Data
(63) Continuation-in-part of application No. 11/132,670, filed on May 19, 2005.

(51) Int. Cl. A61K 9/68; A23G 3/00; A61K 36/899; A61K 36/55

(52) U.S. Cl. 424/440; 424/750; 424/768; 424/776; 426/660

(57) ABSTRACT

The present invention relates to orally ingestible compositions also referred to herein as confectionery products for delivery of saliva stimulating and/or saliva substitute to the mouth and throat. The orally ingestible compositions include a core and a shell surrounding the core so that the core is generally centrally or substantially centrally positioned therein. The composition of the core and shell make the confection uniquely suited for the targeted delivery of the saliva stimulating or saliva substitute to the mouth and throat.

This application is a continuation in part of U.S. patent application Ser. No. 11/132670, filed May 19, 2005.

### FIELD OF THE INVENTION

The present invention relates to orally ingestible compositions, optionally confectionery products, for delivery of saliva stimulating agent and/or saliva substitute to the mouth and throat. By and large, the orally ingestible compositions include a core and a shell surrounding the core so that the core is generally centrally or substantially centrally positioned therein. The composition of the core and shell make the confection uniquely suited for the targeted delivery of the saliva stimulating and/or saliva substitutes to the mouth and throat.

### BACKGROUND OF THE INVENTION

Xerostomia (dry mouth syndrome) is typically the result of compromised salivary flow and can be associated with a wide variety of conditions and causative agents. The condition can also stem from such pathological states as Sicca Syndrome (Sjogren's disease), dry gland disease, polyglandular failure disfunction, and the like. According to a 1986 National Institute of Dental Research publication, over 300 commonly used drugs listed dry mouth as a side effect of their use, the most prevalent of which were the hypertensives and anti-depressants, while others included pain killers, tranquilizers, diuretics and even over-the-counter antihistamines. Furthermore, age and stress have been linked to xerostomia.

As mentioned above, compromised salivary flow is frequently responsible for a variety of dry mouth symptoms which include a burning sensation in the mouth, sleeping difficulties, difficulty with speech, difficulty eating and tasting and the like. It can also lead to more serious disorders such as mucosal infections, bacterial sialadentis, periodontal disease and dental caries.

At present, dry mouth sufferers have used a variety of self-help treatments. Liquid remedies such as water and artificial saliva offer short lived help, and the artificial saliva exhibits a consistency similar to natural saliva which some sufferers view as distasteful. Solid aids such as citric rinds and hard candies have also been tried. In light of the above mentioned difficulties, a continuing need exists for new or improved products useful in helping dry mouth suffers prevent or reduce the occurrence of the symptoms associated with xerostomia.

Accordingly, it would be desirable to provide orally ingestible compositions capable of providing both saliva stimulating and saliva substitutes to the oral cavity. Further, it would be desirable to provide a “center-fill” orally ingestible composition that is capable of delivering saliva stimulating or saliva substitutes to the mouth and throat that reduce the unpleasant taste or mouth-feel associated with conventional saliva substitutes. Still further it would be desirable for a lozenge to deliver multiple ingredients at different stages of the administration or agent delivery process.

### SUMMARY OF THE INVENTION

The compositions of the present invention relate to orally ingestible products for the delivery of at least one active for preventing, reducing or alleviating the symptoms of dry mouth comprising:

a) a water dissolvable or erodible shell comprising a saliva stimulating agent; and

b) a core component comprising:
i.) a saliva substitute; and
ii.) optionally, a saliva stimulating agent.

Methods of using the above compositions are also disclosed.

### DETAILED DESCRIPTION OF THE INVENTION

The orally ingestible compositions of the present invention can comprise, consist of, or consist essentially of the essential elements and limitations of the invention described herein, as well any of the additional or optional ingredients, components, or limitations described herein.

All percentages, parts and ratios are based upon the total weight of the orally ingestible composition of the present invention, unless otherwise specified.

By the term “safe and effective amount” as used herein means an amount of a compound or composition such as a topical or system active (or formulation) sufficient to significantly induce a positive benefit, for example, dry mouth relief, including independently the benefits disclosed herein, but low enough to avoid serious side effects, i.e., to provide a reasonable benefit to risk ratio, within the scope of sound judgment of the skilled artisan.

Edible Shell

The compositions of the present invention comprise a water dissolvable or erodible, edible shell. The phrase “water dissolvable or erodible” as used herein, means a shell that can dissolve or erode fast or slowly in the environment of the oral cavity such that the core component is released into the oral cavity. The edible shell can be a chewing gum or a hard or soft candy. The edible shell can also take the form of a capsule or microcapsule. Examples of Center-filled chewing gums can be found in U.S. Pat. No. 3,894,154, herein incorporated by reference in its entirety. Center-filled hard candies are described in U.S. Pat. No. 4,372,942 and U.S. Pat. No.4,466,983, each of which are incorporated by reference in their entirety.

The inner surface of the shell can also have a separate edible lining to prevent or reduce interaction of the filling with the edible shell. The edible shell can also further comprise flavors as described in further detail below. Examples of edible shells suitable for use in the compositions of the present invention can be found in U.S. Pat. Nos. 6,238,690 issued May 29, 2001; 5,795,590 issued Aug. 18, 1998; and 5,595,757 issued Jan. 21, 1997; all to Kiefer et al. and U.S. Pat. No. 5,300,305 issued Apr. 5, 1994 to Stapler et al.; each of which patents is herein incorporated by reference in its entirety.

In certain embodiments, the edible shell comprises a saliva stimulating agent. Saliva stimulating agents suitable for use in the present invention, include, but are not limited to, fruit acids or an acid component such as phosphoric acid, adipic acid, succinic acid, citric acid, malic acid, tartaric acid, fumaric acid, lactic acid, acetic acid, cinnamic acid and mixtures thereof. Without being limited by theory, it is believed that in addition to its saliva stimulating effect, the saliva stimulating agent also assists in the taste-masking of the saliva substitute. The saliva stimulating agent can be present in the edible shell at concentration of from about 0.1% to about 10% by weight of the edible shell, optionally from about 1% to about 5%, and optionally about 1.5% to about 3%. In certain embodiments, the saliva stimulating agents present in the edible shell are citric acid, malic acid, tartaric acid, fumaric acid and mixtures thereof. Saliva stimulating agents are further disclosed in U.S. Pat. No. 4,820,506; herein incorporated by reference in its entirety. Also useful herein is Jambu® manufactured by Takasago as well as anhydrous crystalline maltose such as “FINETOSE”, an anhydrous crystalline maltose commercialized by Hayashibara Biochemical Laboratories, Inc., Okayama, Japan as described in U.S. Pat. No. 6,897,202 to Shibuya et al., herein incorporated by reference in its entirety.

Core Fill Component

The compositions of the present invention also comprise a core comprising a core fill component. In certain embodiments, the core fill component of the present invention comprises at least one saliva stimulating and/or saliva substitute. Other conventional fill component materials can also be present in the core fill component of the present invention.

As used herein, the term “core fill component” includes the combination of a carrier material and an active agent. The term “carrier material”, as used herein, means those ingredients which when combined with the active agent form the core material.

In certain embodiments, the core fill is made of a carrier material which can range in form from a liquid or a liquid-like state to a gel or a gel-like state once exposed to the oral cavity. Prior to delivery of the lozenge to the oral cavity, the core may be in a low viscosity liquid (or liquid-like state), gel (or gel-like state) or a solid state form. At ambient room temperature (24-26° C.), the core may have a viscosity of from about 1,000 to about 100,000 cps, optionally from about 5,000 to about 50000 cps, and optionally from about 10,000 to about 20,000 cps (Brookfield RVT, #4 or #5 RV Spindle, 10 rpm). In certain embodiments, once the confectionery of the present invention is administered to the buccal cavity, the heat of the oral cavity warms the core fill material such that its viscosity is reduced, enabling the core fill component to disperse through out and coat the oral cavity. At oral cavity temperature (30-37° C.), the core fill may have a viscosity from about 1 to about 1,000 cps, optionally from about 20 to about 800 cps, optionally from about 100 to about 500 cps (Brookfield RVT, #4 or #5 RV Spindle, 10 rpm).

In certain embodiments, once the orally edible composition of the present invention is administered to the buccal cavity, the heat of the oral cavity warms the core so that it is in a less viscous liquid (or, liquid like) to gel (or, gel-like) state thereby enabling the core fill component to disperse through out and coat the oral cavity. In one embodiment, the core material forms a liquid when exposed to the oral cavity. In certain embodiments, the amount of the core material relative to the confectionery product will typically be in the range of up to about 75% by weight, optionally from about 10 to about 40%, optionally from about 20 to about 60% by weight based on the total weight of the confectionery product.

The core fill component and the edible shell may include any material, which is compatible with the formation of a carrier material suitable for delivery and/or dispersion of the saliva stimulating agent or saliva substitute throughout the oral cavity. In particular, useful materials include sweeteners as described below, emulsifiers such as lecithin and the like, taste masking agents such as aluminum magnesium silicate and the like; and fats and oils such as medium chain triglycerides, such as Neobee 1053 as sold by Stepan may be used in the shell and/or as part of the core component. Flavorings such as menthol, eucalyptol, strawberry flavorings such as those sold by Firmenich and the like. Additional details and examples of these materials are described in U.S. Pat. No. 6,596,298 to Leung et al., herein incorporated by reference in its entirety.

In certain embodiments, the core fill component comprises, in addition to the edible shell, at least one saliva stimulating agent. The saliva stimulating agents previously mentioned are also useful in the core fill component of the present invention. When present in the core fill component, the saliva stimulating agent can be present at concentration of from about 0% to about 10% by weight of the core fill component, optionally from about 0.1% to about 5%, optionally about 1% to about 3%.

In certain embodiments, the saliva stimulating agents present in the core fill the component are citric acid, malic acid, tartaric acid, fumaric acid and mixtures thereof. Additionally, anticholinergic agents such as pilocarpine as described U.S. Pat. No. 6,200,551 to Morgan, issued Mar. 13. 2001 and cholinesterase inhibitors as described in U.S. Pat. No. 5,962,503 to Ekstrom et al. can be used in the core, both of which listed patents are herein incorporated by reference in their entirety.

In certain embodiments, the core fill component comprises a saliva substitute or replacement agent. Saliva substitutes suitable for use in the core fill component of the present invention include, but are not limited to, linseed polysaccharide base compounds, oat or oat derived materials, carboxymethylcellulose and mineral salts mixtures, chitins or chitosans, synthetic polyalkylene oxide solutions or mixtures thereof.

By “linseed polysaccharide base compounds” are meant compounds as disclosed in U.S. Pat. No. 5,260,282 to Attstrom et al., herein incorporated by reference in its entirety. These compounds are available under the trade name Salinum, Sinclair Pharmaceuticals Ltd, U.K By “oat or oat derived materials” are meant compounds such as colloidal oatmeal, oat extract, oat based proteins (e.g., gluten), oat based carbohydrates (e.g., xylan, maltodextrin [e.g., Oatrim-5], glucans), oat based surfactants or mixtures thereof as described in U.S. Pat. Appl. 2001047157 to Burnett et al., published Nov. 29, 2001; US Pat Appl. 20010011083 to Barr et al., published Aug. 2, 2001; US Pat. Appl. 20040086547 to Prosise et al., published May 6, 2004; U.S. Pat. No. 6,168,799 to Klein et al., issued Jan. 2, 2001; U.S. Pat. No. 6,753,020 to Mayne, issued Jun. 22, 2004; U.S. Pat. No. 6,736,266 to Flaig et al., issued May 18, 2004; U.S. Pat. No. 5,399,350 to Potter, issued Mar. 21, 1995; U.S. Pat. No. 6,113,964 to Potter, issued Sep. 5, 2000; and U.S. Pat. No. 6,464,991 to Walele et al., issued Oct. 15, 2002; all of which patents and patent applications are herein incorporated by reference in their entirety.

Also useful are carboxymethylcellulose and mineral salts mixtures such as products under the trade names XERO-LUBE®, OREX, ® SAL-ESE®, SALIVART®, and MOI-STIR.®; chitins and/or chitosans as disclosed by U.S. Pat. No.4,879,281 to Shibasaki et al.; and synthetic polyalkylene oxide solutions as disclosed in U.S. Pat. No. 3,767,789 to Rankin, each of which patents are herein incorporated by reference in its entirety.

In certain embodiments, the saliva substitute present in the core fill component is selected from the group consisting of linseed polysaccharide base compounds, oat or oat derived materials and mixtures thereof.

The saliva substitute can be present in the fill component at concentrations of from about 1% to about 95% by weight of the core fill component, optionally from about 2% to about 50%, optionally about 5% to about 25%.

The core fill component of the present invention can be a solid, particularly a powder, or a liquid, including forms of intermediate consistency such as a paste or a gel. When the core fill component is an aqueous fill component, water can be present in the core fill component at levels of from about 5% to about 95%, optionally from about 8% to about 20%, optionally from about 10% to about 15% by weight of the fill component.

In certain embodiments, the core is surrounded by the shell of the confectionery product in a manner such that there is little, if any, leaking of the core material into the shell and outside of the confectionery product prior to dissolution. It is desired, but not required, that the core be provided in a definable shape and is visible through the shell. By being visible through the shell of the confectionery product, the resulting product has a visible display of the core containing an active agent.

The core may be provided in any shape. Suitable shapes include such conventional shapes as spheres, cubes, stripes, and the like, including a plurality of the same as well as less conventional or eccentric shapes, such as cartoon characters, polygons, symbols (e.g. a letter of the alphabet) and the like. The ability to fashion the core in a variety of shapes facilitates use of the present invention where children are concerned (e.g. as when the core is in the shape of a cartoon character). Furthermore, the shape of the core may be useful in identifying particular active agents (e.g. a square shape may indicate the presence of a particular antibiotic).

Optional Ingredients

The compositions of the present invention can optionally include in either the edible shell or as core fill components (or both) additional ingredients such as pharmaceutical actives. Pharmaceutical actives useful herein as optional ingredients are described in previously incorporated reference U.S. Pat. No. 6,596,298.

Sweeteners may be incorporated in orally ingestible composition of the present invention as needed for manufacturing purposes or for purposes of taste. For example, sugar, isomalt, glucose, frutose and/or sugar alcohols such as sorbitol, xylitol, mannitol, glycerin or mixtures thereof are useful herein. Additionally or alternatively, so-called artificial sweeteners such as acesulfame K, sucralose, aspartame, saccharin, tagatose or mixtures thereof are also useful in the confectionaries of the present invention. A more detailed discussion of sweeteners can be found in previously incorporated U.S. Pat. No. 6,596,298 to Leung et al.

Additionally, the orally ingestible composition of the present invention can optionally include a flavoring agent. As used herein, the term ‘flavoring agent’ means those natural flavor essences and equivalent synthetic ingredients which are added to the flavor composition for the principal purpose of providing flavor to the confectionery product. Flavoring agents well known in the confectionery art can be added to the flavor compositions of the invention. These flavoring agents can be chosen from synthetic flavoring liquid and/or oils derived from plants leaves, flowers, fruits and so forth, and combinations thereof. Representative flavoring liquids include: artificial, natural or synthetic fruit flavors such as lemon, orange, banana, grape, lime, apricot and grapefruit oils and fruit essences including apple, strawberry, cherry, orange, pineapple and so forth; bean and nut derived flavors such as coffee, cocoa, cola, peanut, almond and so forth; and root derive flavors such as licorice. Also useful as flavors are tea leaf extracts such as extracts from green tea, oolong tea, black tea as described in U.S. Pat. No. 6,726,939 to Pak, issued Apr. 27, 2004, herein incorporated by reference in its entirety. The amount of flavoring agent employed is normally a matter of preference subject to such factors as flavor type, base type and strength desired. In general, amounts up to about 4% by weight of the total composition are usable with amounts of from about 0.1% to about 1% being optional.

Vitamins are also optionally useful in the present compositions. Examples of vitamins suitable for the incorporation in the composition of the invention include Vitamin A, Vitamin D, Vitamin E, Vitamin K, Vitamin C, folic acid, thiamin, riboflavin, Vitamin B(6), Vitamin B(12), niacin, biotin and pantothenic acid or pantothenic acid derivatives such as panthenol in pharmaceutical or nutritionally acceptable form. Examples of mineral elements and trace elements suitable for the incorporation in the composition of the invention include calcium, sodium, potassium, phosphorous, magnesium, manganese, copper, zinc, iron, selenium, chromium and molybdenum in pharmaceutical or nutritionally acceptable forms. Such mineral elements can be supplied as sodium chloride, potassium chloride, sodium bicarbonate, monobasic potassium phosphate, dibasic potassium phosphate and mixtures thereof. Useful amounts of a vitamin include from about 0.05 mg to about 3000 mg depending on the vitamin.

Optionally, the compositions of the present invention may further include one or more antimicrobial agents. Suitable antimicrobial agents include, but are not limited to, essential oils. Essential oils are volatile aromatic oils which may be synthetic or may be derived from plants by distillation, expression or extraction, and which usually carry the odor or flavor of the plant from which they are obtained. Useful essential oils may provide antiseptic activity. Some of these essential oils also act as flavoring agents. Useful essential oils include but are not limited to thymol, menthol, methyl salicylate (wintergreen oil), eucalyptol, carvacrol, camphor, anethole, carvone, eugenol, isoeugenol, limonene, osimen, n-decyl alcohol, citronel, a-salpineol, methyl acetate, citronellyl acetate, methyl eugenol, cineol, linalool, ethyl linalaol, safrola vanillin, spearmint oil, peppermint oil, lemon oil, orange oil, sage oil, rosemary oil, cinnamon oil, pimento oil, laurel oil, cedar leaf oil, gerianol, verbenone, anise oil, bay oil, benzaldehyde, bergamot oil, bitter almond, chlorothymol, cinnamic aldehyde, citronella oil, clove oil, coal tar, eucalyptus oil, guaiacol, tropolone derivatives such as hinokitiol, avender oil, mustard oil, phenol, phenyl salicylate, pine oil, pine needle oil, sassafras oil, spike lavender oil, storax, thyme oil, tolu balsam, terpentine oil, clove oil, and combinations thereof. In one embodiment the essential oils are selected from thymol, methyl salicylate, eucalyptol, menthol and combinations thereof.

Other materials which may be incorporated in the present invention include viscosity modifying agents, taste masking agents, demulcents (i.e. throat coating agents) and the like as well as mixtures thereof.

Suitable examples of viscosity modifying agents include medium chain triglycerides, glycerin, emulsifiers (e.g. lecithin, polysorbate 80, mono and diglycerides and the like), propylene glycol, benzyl alcohol, triacetin, carboxiinides, and mixtures thereof as described in previously incorporated by reference U.S. Pat. No. 6,596,298.

Taste masking agents useful herein include fats and oils (e.g. partially hydrogenated cottonseed oil, hydrogenated coconut oils, aloe vera extracts, glycyrrhizin salts such as dipotassium glycyrrhizin and mixtures thereof.

Demulcents useful herein include pectin, glycerin, gelatin and gums such as carrageenan, locust bean, guar, xanthan and gellan and the like and mixtures thereof.

Preparation of the Center Fill Compositions

The orally ingestible compositions of the present invention can be prepared in a manner such that the core fill component in the core is surrounded by the shell. In one embodiment of the invention, the shell is either transparent or translucent allowing the core and/or core fill component to be visible through the shell for “showing” the user the presence of the core fill component in the core. The visible core or core fill component can further be formed into a designated shape matched to a particular active agent present in the core fill component (as previously described). In another embodiment of the invention, the shell is essentially clear (i.e. transparent or translucent and colorless) to provide the greatest contrast between the shell and a colored core fill component. The core or the core fill component may be any color depending on the selection of a food grade suitable coloring agent. Examples of typical coloring agents include FD&C Red 40, FD&C Blue 2, Carmine, FD&C yellow 5, beta carotene and the like.

A shell which enables the core to be seen through the shell can be prepared, for example, in the following manner. An isomalt slurry is quickly heated such as by microfilm cooking techniques. Quick cooking (e.g. for about 5 minutes) minimizes browning of the Isomalt thus creating a very clear shell. A small of amount of coloring agent or saliva stimulating agent may be added if desired.

The confectionery products of the present invention can also be in the form of a lozenge or a hard sucking candy but may include lollypops, and any other shaped or formed product which can be formed from a core fill component materials and edible shell materials in accordance with the present invention.

The confectionery products of the present invention can be prepared using a variety of processing technologies including double depositing, hand-pressing, rotary forming and extrusion. Such techniques are well known in the art such as disclosed in Sugar Confectionery Manufacture, 2^{nd} Edition, Edited by E. B. Jackson (1995), incorporated herein by reference in its entirety. In an embodiment of the invention, the confectionery product is made by separately combining the ingredients of the shell and the core in a vessel and then delivering a stream of the respective materials to a manifold which provides for the interruptible flow of the core ingredients and a continuous flow of the shell ingredients surrounding the core. The resulting product is ejected in discrete units corresponding to the desired weight and size of the confectionery product and placed in trays with individual compartments for storing the confectionery products until they cool to ambient temperature.

In one embodiment of the present invention, the core fill component ingredients are degassed. Degassing techniques remove air from the core material thus at least minimizing chemical reactions therein. The core can be prepared in an enclosed mixing vessel and processed under vacuum. Alternatively, the core fill component ingredients are combined and mixed together and then a vacuum is applied to the mixture to remove any gases contained therein.

In one embodiment, a process for forming the confectionery product is used where the core or core fill components are directly injected within the shell. One such valve system is a manifold system, which may employs a ball/stall or ball/spring valve assembly. This ensures that the core completely surrounded by the shell and allows the core to be deposited within the final product (e.g. lozenge).

The process is typically temperature controlled with a series of heaters/coolers shown sufficient to maintain the shell at a temperature of from about 1° C. to about 200° C. and the core material from about 1° C. to about 200° C. which is a temperature sufficient to maintain the core material centrally positioned within the shell material and to enable the same to be ejected as discrete units of the confectionery product.

### EXAMPLES

The center-fill lozenge compositions illustrated in following examples illustrate specific embodiments of the center-fill lozenge compositions of the present invention, but are not intended to be limiting thereof. Other modifications can be undertaken by the skilled artisan without departing from the spirit and scope of this invention.

Method of Preparation

Shell Preparation

The Isomalt and water are added and mixed in a suitable vessel under heating to about 165° C. to form a candy base. A suitable saliva stimulating agent (e.g., citric acid, malic acid, tartaric acid etc.,) is then added to the vessel. The candy base is then cooled to about 145° C. A suitable sweetener (e.g. a high intensity sweetener such as acesufame K, aspartame, neotame and the like or mixtures thereof) can then be added along with any optional active agents and flavors and any other suitable ingredients.

Center Preparation

The core material may be prepared by mixing maltitol syrup (Lycasin 80/55 from Roquette America), saliva substitute or replacement agent and, if desired, a colorant in a suitable vessel under heating to form a candy base. The candy base is then cooled to about 70° C. or lower to enable the addition of a beta carotene, a suitable viscosity modifying agent, such as glycerin, sweetener (e.g. high intensity sweetener) the active agent, a flavorant and any other suitable ingredients.

The respective shell and core materials are then added to separate hoppers which materials are then combined as previously described.

### Example I

### A Centerfilled Lozenge for Dry Mouth with Linseed Extract in Center

A center-filled lozenge for dry mouth having a core containing linseed extract is prepared according to the above Method of Preparation and had a formulation as specified below.

| | | Center- | |
|---|---|---|---|
| | | Fill | |
| | Shell | % | Total |
| | % | w/w in | Product |
| | w/w in | Center- | % |
| Ingredient | Shell | Fill | w/w |
| Isomalt¹ | 93.9816 | 0.0000 | 81.4507 |
| Purified | 0.6300 | 0.0000 | 0.5460 |
| Water | | | |
| Citric Acid | 0.0500 | 0.0000 | 0.0433 |
| Acesulfame | 0.0340 | 0.0000 | 0.0295 |
| Potassium | | | |
| Salt | | | |
| Aspartame | 0.0680 | 0.0000 | 0.0589 |
| Orange | 0.2000 | 0.0000 | 0.1733 |
| Flavor | | | |
| Lycasin | 0.0000 | 79.9636 | 10.6618 |
| (maltitol | | | |
| syrup), | | | |
| Roquette | | | |
| Beta Carotene | 0.0352 | 0.0352 | 0.0352 |
| 2% WD | | | |
| Emulsion, 3030 | | | |
| Color | 0.0012 | 0.0012 | 0.0012 |
| Salinun | 5.0000 | 20.0000 | 7.0000 |
| (linseed | | | |
| extract)² | | | |
| | 100.0000 | 100.0000 | 100.0000 |

¹Hydrogenated isomalt, supplied by Palatinit of America. The % Isomalt in the finished product refers to amount of Cooked Isomalt which will contain about 1.5% moisture.

### Example II

A Centerfilled Lozenge for Dry Mouth with an Oatmeal Extract in Center

A center-filled lozenge for dry mouth having a core containing an oatmeal extract is prepared according to the above Method of Preparation and had a formulation as specified below.

| | | Center- | Total |
|---|---|---|---|
| | Shell | Fill | Product |
| | % | % | % |
| | in | in | in |
| | finished | finished | finished |
| Ingredient | shell | center | lozenge |
| Cooked | 98.9816 | 0.0000 | 85.7841 |
| Isomalt¹ | | | |
| Purified | 0.6300 | 0.0000 | 0.5460 |
| Water² | | | |
| Malic Acid | 0.0500 | 0.0000 | 0.0433 |
| Acesulfame | 0.0340 | 0.0000 | 0.0295 |
| Potassium | | | |
| Salt | | | |
| Aspartame | 0.0680 | 0.0000 | 0.0589 |
| Orange | 0.2000 | 0.0000 | 0.1733 |
| Flavor | | | |
| Lycasin | 0.0000 | 79.9636 | 10.6618 |
| (maltitol | | | |
| syrup), | | | |
| Roquette | | | |
| Beta | 0.0352 | 0.0352 | 0.0352 |
| Carotene 2% | | | |
| WD | | | |
| Emulsion, | | | |
| 3030 | | | |
| Color | 0.0012 | 0.0012 | 0.0012 |
| Oat beta | 0.0000 | 20.0000 | 2.6667 |
| glucan (FG | | | |
| grade)² | | | |
| | 100.0000 | 100.0000 | 100.0000 |

¹Hydrogenated isomalt, supplied by Palatinit of America. The % Isomalt in the finished product refers to amount of Cooked Isomalt which will contain about 1.5% moisture.
²Supplied by Ceapro Inc, a subsidiary of Symrise company, Germany

### Example III

### A centerfilled Lozenge for Dry Mouth with Carboxymethylcellulose in Center

A center-filled lozenge for dry mouth having a core containing carboxymethylcellulose is prepared according to the above Method of Preparation and had a formulation as specified below.

| | | Center- | Total |
|---|---|---|---|
| | Shell | Fill | Product |
| | % | % | % |
| | in | in | in |
| | finished | finished | finished |
| Ingredient | shell | center | lozenge |
| Cooked Isomalt¹ | 95.9816 | 0.0000 | 83.1841 |
| Purified Water² | 0.6300 | 0.0000 | 0.5460 |
| Tartaric Acid | 0.0500 | 0.0000 | 0.0433 |
| Acesulfame Potassium | 0.0340 | 0.0000 | 0.0295 |
| Salt | | | |
| Aspartame | 0.0680 | 0.0000 | 0.0589 |
| Carrubba Orange Flavor | 0.2000 | 0.0000 | 0.1733 |
| D5580 | | | |
| Lycasin (maltitol syrup), | 0.0000 | 79.9636 | 10.6618 |
| Roquette | | | |
| Beta Carotene 2% WD | 0.0352 | 0.0352 | 0.0352 |
| Emulsion, 3030 | | | |
| Sodium Chloride | 3.0000 | 0.0000 | 2.6000 |
| FD&C Red 40 Dye | 0.0012 | 0.0012 | 0.0012 |
| Carboxymethylcellulose | 0.0000 | 20.0000 | 2.6667 |
| | 100.0000 | 100.0000 | 100.0000 |

¹Hydrogenated isomalt, supplied by Palatinit of America. The % Isomalt in the finished product refers to amount of Cooked Isomalt which will contain about 1.5% moisture.

### Example I

### A Centerfilled Lozenge for Dry Mouth with Linseed Extract in Center

A center-filled lozenge for dry mouth having a core containing linseed extract is prepared according to the above Method of Preparation and had a formulation as specified below.

| | | Center- | |
|---|---|---|---|
| | | Fill | |
| | Shell | % | Total |
| | % | w/w in | Product |
| | w/w in | Center- | % |
| Ingredient | Shell | Fill | w/w |
| Isomalt¹ | 88.9816 | 0.0000 | 81.4507 |
| Purified | 0.6300 | 0.0000 | 0.5460 |
| Water | | | |
| Citric Acid | 0.0500 | 0.0000 | 0.0433 |
| Acesulfame | 0.0340 | 0.0000 | 0.0295 |
| Potassium | | | |
| Salt | | | |
| Aspartame | 0.0680 | 0.0000 | 0.0589 |
| Orange | 0.2000 | 0.0000 | 0.1733 |
| Flavor | | | |
| Lycasin | 0.0000 | 74.9636 | 10.6618 |
| (maltitol | | | |
| syrup), | | | |
| Roquette | | | |
| Beta Carotene | 0.0352 | 0.0352 | 0.0352 |
| 2% WD | | | |
| Emulsion, 3030 | | | |
| Color | 0.0012 | 0.0012 | 0.0012 |
| Salinum | 10.0000 | 25.0000 | 7.0000 |
| (linseed | | | |
| extract)² | | | |
| | 100.0000 | 100.0000 | 100.0000 |

¹Hydrogenated isomalt, supplied by Palatinit of America. The % Isomalt in the finished product refers to amount of Cooked Isomalt which will contain about 1.5% moisture.

What is claimed is:

1. A confectionery product for the delivery of at least one active for preventing, reducing or alleviating the symptoms of dry mouth comprising:
a) a water dissolvable or erodible shell comprising a saliva stimulating agent; and
b) a core component comprising:
i.) a saliva substitute; and
ii.) optionally, a saliva stimulating agent.

2. A confectionery product according to claim 1, wherein the saliva substitute is selected from the group consisting of linseed polysaccharide base compounds, tamarind seed polysaccharide base compounds, oat or oat derived materials, carboxymethylcellulose and mineral salts mixtures, chitins or chitosans, synthetic polyalkylene oxide solutions, and mixtures thereof.

3. A confectionery product according to claim 2, wherein the saliva substitute is selected from the group consisting of linseed polysaccharide base compounds, tamarind seed polysaccharide base compounds, oat or oat derived materials, and mixtures thereof.

4. A confectionery product according to claim 1, wherein the saliva stimulating agent is an acid.

5. A confectionery product according to claim 4, wherein the saliva stimulating agent is a fruit acid.

6. A confectionery product according to claim 5, wherein the saliva stimulating agent is a fruit acid selected from the group consisting of phosphoric acid, adipic acid, succinic acid, citric acid, malic acid, tartaric acid, fumaric acid, lactic acid, acetic acid, cinnamic acid, and mixtures thereof.

7. A confectionery product according to claim 6, wherein the saliva stimulating agent is fruit acid selected from the group consisting of, citric acid, malic acid, tartaric acid, fumaric acid, and mixtures thereof.

8. A confectionery product according to claim 3, wherein the saliva substitute is a linseed polysaccharide base compound.

9. A confectionery product according to claim 3, wherein the saliva stimulating agent is an acid.

10. A confectionery product according to claim 10, wherein the saliva stimulating agent is a fruit acid.

11. A confectionery product according to claim 3, wherein the saliva substitute is a tamarind seed polysaccharide base compound.

12. A confectionery product according to claim 11, wherein the saliva stimulating agent is an acid.

13. A confectionery product according to claim 12, wherein the saliva stimulating agent is a fruit acid.

14. A confectionery product according to claim 3, wherein the saliva substitute is an oat or oat derived material.

15. A confectionery product according to claim 14, wherein the saliva stimulating agent is an acid.

16. A confectionery product according to claim 15, wherein the saliva stimulating agent is a fruit acid.

17. A confectionery product according to claim 1, further comprising anticholinergic agents, cholinesterase inhibitors, and mixtures thereof.

18. A confectionery product according to claim 8, further comprising anticholinergic agents, cholinesterase inhibitors, and mixtures thereof.

19. A confectionery product according to claim 11, further comprising anticholinergic agents, cholinesterase inhibitors, and mixtures thereof.

20. A confectionery product according to claim 14, further comprising anticholinergic agents, cholinesterase inhibitors, and mixtures thereof.
